(19) United States
(12) Patent Application Publication
(10) Pub. No.: US 2008/0160549 A1
(43) Pub. Date: Jul. 3, 2008

(54) Immunochromatography kit

(75) Inventors: Takayoshi Oyamada, Kanagawa (JP); Junichi Katada, Kanagawa (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 11/979,849

(22) Filed: Nov. 8, 2007

(30) Foreign Application Priority Data
Nov. 8, 2006 (JP) 2006-302843

(51) Int. Cl. G01N 33/00

(52) U.S. Cl. 435/7.94

(57) ABSTRACT

An immunochromatography kit including an inorganic silver salt or a silver complex, a reducing agent for silver ions, and a metal colloid label or a metal chalcogenide label. The immunochromatography kit uses an immune reaction of an analyte and an antibody or antigen that can specifically bind to the analyte, and analyzes a signal from a label derived from the immobilized immune complex.

### CROSS-REFERENCE TO RELATED APPLICATION

This application claims priority under 35USC 119 from Japanese Patent Application No. 2006-302843, the disclosure of which is incorporated by reference herein.

### BACKGROUND OF THE INVENTION

1. Field of the Invention

The present invention relates to an immunochromatography kit with which a sample containing an analyte can be qualitatively or quantitatively analyzed easily, promptly and accurately.

2. Description of the Related Art

Among biologically active substances or environmental pollutants such as natural products, toxins, hormones and agricultural chemicals, there are numerous substances acting in an ultratrace amount. Accordingly, instrumental analytical methods capable of high-sensitivity analysis have conventionally been widely used for qualitative and quantitative measurement of these substances. However, instrumental analytical methods are poor in specificity, require time for analysis including pretreatment of a sample, and are troublesome in operation. Thus instrumental analytical methods are inconvenient for the purpose of rapid and easy measurements for which there have been needs in recent years. On the other hand, immunological measuring methods are highly specific and much easier in operation than instrumental analytical methods. Therefore immunological measuring methods have gradually spread in the field of measurement of biologically active substances and environmental pollutants. However, conventional immunological measuring methods such as enzyme immunoassays and latex agglutination assays using 96-well plates do not always provide satisfactory rapidness and easiness of measurement or detection sensitivity.

There are also needs for improvement of the sensitivity of tests which currently use relatively invasive samples such swab and blood, the result of which is expected to realize less burdensome tests to patients in which a very small amount of an analyte contained in less invasive samples such as snot, mouth wash and urine is detected.

In recent years, examination kits using immunochromatography (referred to hereinafter as immunochromatography kit) have been used more often in examination of infections which requires particularly rapid diagnosis. According to spread of these kits, patients with infections can be identified using a rapid and easy method, and subsequent diagnosis and therapy can be conducted immediately and accurately. For example, in immunochromatography utilizing the sandwich method, a labeled second antibody capable of binding to an analyte (for example, an antigen), and a sample solution which may possibly contain the analyte, are developed in an insoluble thin film-shaped support (for example, a glass fiber membrane, a nylon membrane or a cellulose membrane) on which a first antibody capable of specifically binding to the analyte was immobilized in a specific region. As a result, an immune complex with the analyte is formed on the region of the insoluble thin film-shaped support which region has the first antibody immobilized thereon. The analyte can be measured by detecting a signal such as color development or coloring of a label. The label may be, for example, a protein such as an enzyme, colored latex particles, metal colloids, or carbon particles.

Immunochromatography does not require any massive facilities or instruments for judgment and measurement. Further, immunochromatography is simple in operation and promptly gives measurement results by dropping a sample solution which may possibly contain an analyte and leaving it for about 5 to 10 minutes. For this reason, this technique is used widely as easy, rapid and highly specific methods of judgment and measurement in many scenes, for example, for clinical examination in hospitals and in assays in laboratories.

Among biologically active substances or environmental pollutants such as natural products, toxins, hormones and agricultural chemicals, there are many substances that are effective even in ultratrace amounts that are undetectable by conventional common immunochromatography. Therefore, there are demands for development of rapid, easy and highly sensitive immunochromatography methods.

A large number of techniques attempting at higher sensitivity have conventionally been disclosed, such as techniques of a innovated means of development (see, for example, Japanese Patent Application (JP-A) No. 1-32169 and JP-A No. 4-299262), techniques of innovated colored particles (see, for example, JP-A No. 5-10950 and JP-A No. 5-133956), techniques of innovated member for development (see, for example, JP-A No. 7-318560), techniques utilizing an avidin-biotin bond (see, for example, JP-A No. 10-68730), techniques utilizing an enzyme immunoassay (see, for example, JP-A No. 11-69996), techniques using catalytically active metal colloids (see, for example, JP-A No. 2003-262638), and techniques of precipitating metal ions (see, for example, JP-A No. 2002-202307).

However there are needs for still higher sensitivity although the immunochromatography approaches the enzyme immunoassays due to increased sensitivity in the detection of an analyte achieved by these techniques.

### SUMMARY OF THE INVENTION

The present invention has been made in view of the above circumstances and provides an immunochromatography kit.

An aspect of the present invention provides an immunochromatography kit including an inorganic silver salt or a silver complex, a reducing agent for silver ions, and a metal colloid label or a metal chalcogenide label. The immunochromatography kit uses an immune reaction of an analyte and an antibody or antigen that can specifically bind to the analyte, and analyzes a signal from a label derived from the immobilized immune complex.

### BRIEF DESCRIPTION OF THE DRAWINGS

FIG. 1 is a plane view which schematically illustrates an embodiment of a comparative immunochromatography kit.

FIG. 2 is a longitudinal sectional view which schematically illustrates a longitudinal sectional view of the immunochromatography kit illustrated in FIG. 1.

FIG. 3 is a longitudinal sectional view which schematically illustrates an immunochromatography kit according to the present invention.

### DETAILED DESCRIPTION OF THE INVENTION

In general, the detection sensitivity of conventional immunochromatography in the case of bacteria is 10⁵ to 10⁷ CFU/ml. The gene amplification method (PCR method) is mentioned as a recent highly sensitive detection method, which has achieved a detection sensitivity of up to 10³ to 10⁴ CFU/ml. However, the PCR method needs massive facilities and instruments and complicated operation. Moreover, the PCR method requires a long time (several hours) until detection, and thus cannot be considered to be an easy and rapid measurement method. If the sensitivity of conventional immunochromatography is heightened by about 1- to 4-digits, examinations which have conventionally been carried out by the PCR method and which has not been easy and rapid, are expected to be conducted easily and rapidly.

There is also demand for higher sensitivity in examination of infections for which immunochromatographic measurement methods have been established. For example, the examination of influenza by immunochromatography has spread widely in recent years as an easy and rapid examination method; however, re-examination is necessary in some cases because the result could be false negative due to poor detection sensitivity when the amount of the virus in an initial stage of infection is relatively low. In general, influenza virus is considered to grow 10-fold in 4 hours. Therefore improvement of the sensitivity by 1-digit, for example, enables the infection to be judged four hours earlier than conventional methods. From the viewpoint of reducing the burden of patients attending a hospital many times, there is demand for an easy and rapid examination method, such as immunochromatography, with higher sensitivity.

1. Immunochromatography

In general, immunochromatography is a technique in which an analyte is measured and determined easily, rapidly and specifically by the following method. That is, a chromatographic carrier having at least one reaction site containing an immobilizing reagent (such as antibody or antigen) capable of binding to the analyte is used as a solid phase. While a dispersion liquid that includes a detection label dispersed therein modified with a reagent capable of binding to the analyte moves chromatographically on the chromatographic carrier as a mobile phase, the analyte binds specifically to the detection label and reaches the reaction site. The analyte-detection label complex binds specifically to the immobilizing reagent at the reaction site. Therefore the detection label is concentrated at the immobilization reagent part only when the analyte is present in a test solution. The presence of the analyte in the test solution is determined qualitatively and quantitatively through visual inspection of the concentrated detection label or detection with a suitable instrument.

The immunochromatography kit according to the present invention includes therein an inorganic silver salt and a reducing agent for silver ions. The signal is amplified by an amplification reaction using the analyte-detection label complex that is bonded to the immobilizing reagent as a core, resulting in achievement of higher sensitivity. According to the invention, it is possible to provide a simpler and quicker high-sensitivity immunochromatography kit that does not require supply, from the outside, of metal ions or a reducing agent solution for amplification, which is required in conventional immunochromatography kits.

2. Specimen

The specimen to be analyzed with the immunochromatography kit according to the present invention is not particularly limited insofar as it is a sample that possibly contains an analyte of interest. The sample may be a biological sample, examples of which include animal (human in particular) body fluids (for example, blood, serum, blood plasma, spinal fluid, lacrimal fluid, sweat, urine, pus, snot or sputum), excrements (for example, feces), organs, tissues, mucosae and skin, swabs that may possibly contain such samples, mouth washes, animals or plants themselves and dried materials thereof.

3. Pretreatment of the Specimen

The specimen to be examined with the immunochromatography kit according to the present invention may be an intact specimen, or in the form of an extract obtained by extracting the specimen with a suitable extraction solvent, or in the form of a diluted solution obtained by diluting the extract with a suitable diluent, or in the form of a concentrate obtained by concentrating the extract by a suitable method. The extraction solvent to be used may be, for example, a solvent used in an ordinary immunological analysis method (for example, water, physiological saline or a buffer solution) or a water-miscible organic solvent in which the antigen-antibody reaction can be directly carried out after dilution with a solvent that may be selected from those described above.

4. Constitution

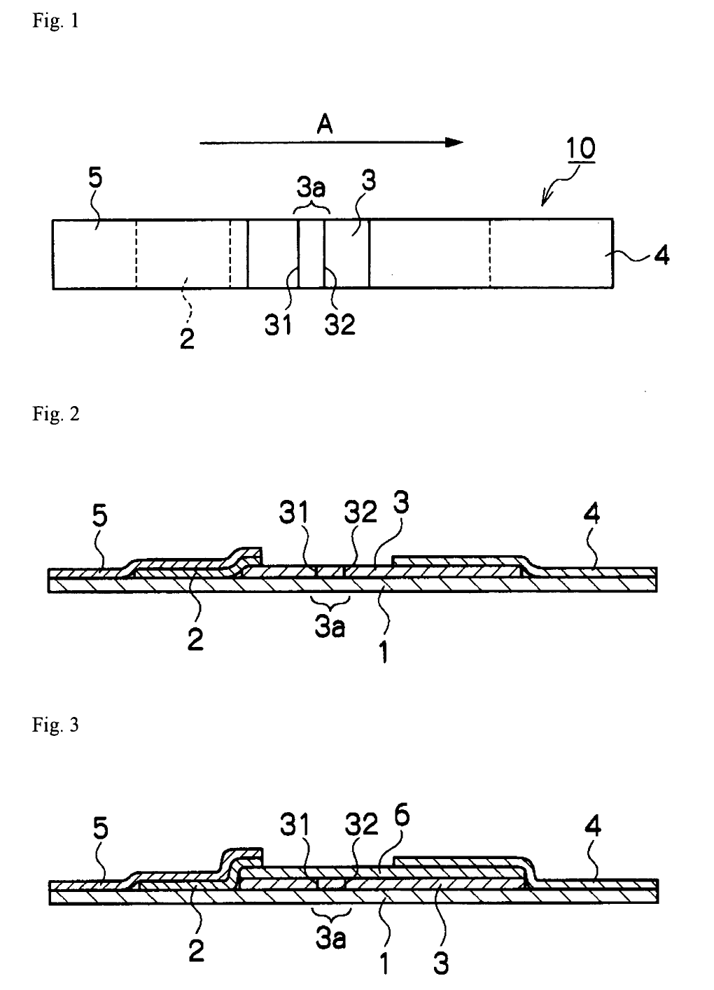

Immunochromatographic strips usable in the immunochromatography kit according to the present invention is not particularly limited insofar as they are immunochromatographic strips usable in ordinary immunochromatography. By way of example, FIG. 1 is a schematic plane view of a conventional immunochromatographic strip. FIG. 2 is a schematic longitudinal sectional view of the immunochromatography kit shown in FIG. 1. FIG. 3 is a schematic sectional view of the immunochromatographic strip according to the invention.

In the immunochromatographic strip 10 according to the invention, a sample addition pad 5, a labeled substance-holding pad (for example, a gold colloid antibody-holding pad) 2, a chromatographic carrier (for example, an antibody-immobilized membrane) 3, and an absorbent pad 4 are arranged, from the upstream to downstream in the direction of development (direction indicated by arrow A in FIG. 1), on a pressure-sensitive adhesive sheet 1.

The chromatograph carrier 3 has a capture site 3a, and has a detecting zone (also referred to as a detection part) 31 that is a region where an antibody or antigen capable of specifically binding to the analyte is immobilized. If desired, the carrier 3 may further have a control zone (also referred to as a control part) 32 that is a region where a control antibody or antigen is immobilized. Furthermore, the detecting zone 31 and the control zone 32 contain an inorganic silver salt for amplification and a reducing agent for silver ions.

The labeled substance-holding pad 2 may be prepared by preparing a suspension containing a label, then applying the suspension onto a suitable absorbent pad (for example, a glass fiber pad) followed by drying.

For example, a glass fiber pad can be used as the sample addition pad 5.

4-1. Detection Label

The detection label may be colored particles used for immune agglutination reaction. For example, latex colored particles of an organic polymer such as polystyrene or a styrene-butadiene copolymer, or a metal or a metal chalcogenide such as metal colloid may be used. The average particle diameter of the carrier particles (or the colloid) is preferably from 0.02 to 10 μm. Liposome or microcapsules containing a colorant may be used as the colored particles. Any conventionally known colored metal colloid may be used as the labeling colored particles. Examples thereof include gold colloids, silver colloids, platinum colloids, iron colloids, aluminum hydroxide colloids, and composite colloids thereof. Preferable examples include gold colloids, silver colloids, platinum colloids, and composite colloids thereof. In particular, gold colloids and silver colloids are preferable in that the gold colloids at a suitable particle diameter show red color and silver colloids at a suitable particle diameter show yellow color. The average particle diameter of a metal colloid is preferably from about 1 nm to about 500 nm, more preferably from 5 nm to 100 nm at which a particularly strong color tone may be obtained. Binding of the metal colloid to the specifically binding substance may be conducted by a method known in the art (for example, The Journal of Histochemistry and Cytochemistry, Vol. 30, No. 7, pp. 691-696 (1982)). That is, the metal colloid and the specifically binding substance (for example an antibody) are mixed in a suitable buffer solution at room temperature for 5 minutes or more. After the reaction, the precipitate obtained by centrifugation is dispersed in a solution containing a dispersant such as polyethylene glycol, whereby the desired metal colloid-labeled specific binding substance can be obtained. When gold colloid particles are used as the metal colloid, commercially available gold colloid particles may be used. As an alternative, gold colloid particles may be prepared by a common method, for example a method of reducing chlorauric acid with sodium citrate (Nature Phys. Sci., vol. 241, 20 (1973) etc.).

According to the present invention, in an immunochromatography kit using, as the detection label, a metal colloid label, a metal chalcogenide label or another metal alloy label (also referred to hereinafter as a metallic label) or a metal-containing polymer particle label, the signal from the metallic label can be amplified. Specifically, after the formation of a complex of the analyte and the detection label, silver ions supplied from an inorganic silver salt and a reducing agent for silver ions are contacted therewith; as a result, the silver ions are reduced with the reducing agent to form silver particles, which deposit on the metallic label as a core, whereby the metallic label is amplified to enable high-sensitivity analysis of the analyte. Accordingly conventionally known immunochromatography can be applied, as it is, to the immunochromatography kit according to the invention except that the precipitation reaction of silver particles generated by reduction of silver ions with the reducing agent is carried out on the label of the immune complex so as to analyze thus amplified signal.

In the immunochromatography kit according to the present invention, a metal colloid label or a metal chalcogenide label may be used as the label for labeling an antibody or antigen which can specifically bind to an analyte (antigen or antibody), or for labeling a standard compound. The metal colloid label or the metal chalcogenide label is not particularly limited as long as the label is usable in an ordinary immunochromatograph method. The metal colloid label may be, for example, a colloid of gold, silver, platinum, palladium, lead, zinc, cadmium, tin, chromium, copper or cobalt, and is preferably a colloid of gold, silver, platinum or palladium, or a mixture thereof. The metal chalcogenide label may be, for example, a sulfide, selenide, or telluride of mercury, copper, gold, silver, platinum, palladium, lead, zinc, nickel, cadmium, tin, chromium, copper or cobalt. In the immunochromatography kit according to the invention, at least one of these metal colloid labels and metal chalcogenide labels may be used as the label.

4-2. Antibody

In the immunochromatography kit according to the present invention, the antibody having specificity for an analyte is not particularly limited; for example, it is possible to use an antiserum prepared from serum of an animal immunized with the analyte, an immunoglobulin fraction purified from the antiserum, a monoclonal antibody obtained by cell fusion using splenocytes of the animal immunized with the analyte, or fragments thereof (for example, F(ab′)2, Fab, Fab′ or Fv). Preparation of such an antibody may be carried out by a common method.

4-3. Chromatographic Carrier

The chromatographic carrier is preferably a porous carrier, particularly preferably a nitrocellulose membrane, a cellulose membrane, an acetyl cellulose membrane, a polysulfone membrane, a polyether sulfone membrane, a nylon membrane, glass fibers, a nonwoven fabric, a cloth, threads or the like.

Usually, a substance for detection is immobilized in a part of the chromatographic carrier to form a detection zone. The substance for detection may be directly immobilized through physical or chemical bonds onto a part of the chromatographic carrier; as an alternative, the substance for detection may be bound physically or chemically onto fine particles such as latex particles and then immobilized by trapping the fine particles onto a part of the chromatographic carrier. Prior to use, the chromatographic carrier after immobilizing the substance for detection thereon is preferably subjected to a treatment for preventing unspecific adsorption. The treatment may be conducted by using an inert protein, etc.

4-4. Sample Addition Pad

Examples of the materials for the sample addition pad include, but are not limited to, those having uniform characteristics, such as a cellulose filter paper, glass fibers, polyurethane, polyacetate, cellulose acetate, nylon and a cotton cloth. The sample addition part not only functions to receive the analyte-containing sample that is added, but also functions to filter off insoluble particles etc. in the sample. The material constituting the sample addition part may be used after being subjected to treatment for preventing unspecific adsorption in order to prevent deterioration in analysis accuracy due to unspecific adsorption of the analyte in the sample onto the material of the sample addition part.

4-5. Labeled Substance Holding Pad

Examples of the material of the labeled substance holding pad include, for example, a cellulose filter paper, glass fibers and a nonwoven fabric. The labeled substance holding pad is prepared by impregnating the pad with a predetermined amount of the detection label prepared as described above, followed by drying.

4-6. Absorbent Pad

The absorbent pad constitutes a portion where the added sample is physically absorbed due to chromatographic migration and where an unreacted label etc. that is not immobilized on the detection part of the chromatographic carrier is removed by absorption. The material for the absorbent pad may be a water-absorbing material such as a cellulose filter paper, a nonwoven fabric, a cloth or cellulose acetate. Because the chromatographic speed of the chromatographic leading end of the added sample after reaching the absorbing portion varies depending on the material and size of the absorbent material, an adequate speed for the measurement of the analyte can be set by selection of the absorbent material.

5. Immunoassay Method

Hereinafter, in reference to specific embodiments where the immunochromatography used in the invention is applied to the sandwich method, antibody immobilizing competitive method, antigen immobilizing competitive method and immobilized antigen method, the immunochromatography used in the invention is described in that order.

5-1. Sandwiching Method

In an embodiment where the sandwich method is applied to the immunochromatography kit according to the present invention (hereinafter referred to simply as the sandwich method), the analysis of the analyte may be conducted, for example, in the following procedures though the procedures are not limited thereto. Initially, first and second antibodies having specificity for an analyte (antigen) are prepared in advance by the method described above. In addition, the second antibody is labeled in advance. The first antibody is immobilized on a suitable insoluble thin film-shaped support (for example, a nitrocellulose membrane, a glass fiber membrane, a nylon membrane or a cellulose membrane) and is brought into contact with a test sample (or its extract) that may possibly contain the analyte (antigen), so that an antigen-antibody reaction occurs if the analyte is present in the test sample. This antigen-antibody reaction may be carried out in the same manner as in usual antigen-antibody reaction. During or after the antigen-antibody reaction, the sample is brought into contact further with an excess amount of the labeled second antibody, so that an immune complex of the immobilized first antibody—the analyte (antigen)-labeled second antibody forms when the analyte is present in the sample.

In the sandwich method, after the completion of the reaction of the immobilized first antibody, the analyte (antigen) and the second antibody, the labeled second antibody not involving in the formation of the immune complex is removed. Subsequently, for example, the signal from the label of the labeled second antibody involved in the immune complex may be amplified by supplying metal ions and a reducing agent to that region of the insoluble thin film-shaped support on which the immobilized first antibody is immobilized. As an alternative, the signal from the label of the labeled second antibody involved in the immune complex may be amplified by adding metal ions and a reducing agent to the labeled second antibody, and adding the labeled second antibody, the metal ions and the reducing agent simultaneously to the thin film-shaped support.

5-2. Antibody Immobilizing Competitive Method

In an embodiment where the antibody immobilizing competitive method is applied to the immunochromatography kit according to the present invention (hereinafter referred to simply as the antibody immobilizing competitive method), the analysis of the analyte may be conducted, for example, in the following procedures though the procedures are not limited thereto. First, an antibody having specificity for an analyte (antigen) is prepared in advance by the method described above, and the antibody is immobilized on a suitable insoluble thin film-shaped support (for example, a nitrocellulose membrane, a glass fiber membrane, a nylon membrane or a cellulose membrane). Separately, the standard compound is labeled in advance. The labeled standard compound and a test sample (or its extract) that may possibly contain the analyte (antigen) are developed and brought into contact with each other, during or after which the labeled standard compound is developed and brought into contact with the immobilized antibody, so that an antigen-antibody reaction occurs if the analyte is present in the sample. This antigen-antibody reaction may be carried out in the same manner as in usual antigen-antibody reaction.

In the antibody immobilizing competitive method, after the reaction of the immobilized antibody on the insoluble thin film-shaped support and the labeled standard compound (that is, the labeled antigen) is completed, the labeled standard compound bonded to the immobilized antibody and the labeled standard compound not bonded to the immobilized antibody are separated from each other. Subsequently, for example, metal ions and a reducing agent may be added to the region of the insoluble thin film-shaped support on which the immobilized antibody is immobilized, so that the signal from the label of the labeled antigen bonded to the immobilized antibody is amplified. As an alternative, metal ions and a reducing agent may be added to the labeled standard compound, and the labeled standard compound, the metal ions and the reducing agent may be simultaneously added to the thin film-shaped support, so that the signal from the label of the labeled standard compound bonded to the immobilized antibody is amplified. The above separation may be carried out for example by washing with a buffer solution.

5-3. Antigen Immobilizing Competitive Method

In an embodiment where the antigen immobilizing competitive method is applied to the immunochromatography kit according to the present invention (hereinafter referred to simply as the antigen immobilizing competitive method), the analysis of the analyte may be conducted, for example, in the following procedures though the procedures are not limited thereto. First, an antibody having specificity for an analyte (antigen) is prepared in advance by the method described above. The antibody is labeled in advance. Further, a known amount of the standard compound (antigen) is immobilized on a suitable insoluble thin film-shaped support (for example, a nitrocellulose membrane, a glass fiber membrane, a nylon membrane or a cellulose membrane).

In the antigen immobilizing competitive method, after completion of the reaction of the immobilized standard compound (that is, the immobilized antigen) on the insoluble thin film-shaped support and the labeled antibody, the labeled antibody bonded to the immobilized standard compound and the labeled antibody not bonded to the immobilized standard compound are separated from each other. Subsequently, for example, the signal from the label of the labeled antibody bonded to the immobilized standard compound may be amplified by supplying metal ions and a reducing agent to the region of the insoluble thin film-shaped support on which the immobilized standard compound is immobilized. As an alternative, metal ions and a reducing agent may be added to the labeled antibody, and the labeled antibody, the metal ions and the reducing agent may be simultaneously added to the thin film-shaped support, so that the signal from the label of the labeled standard antibody bonded to the immobilized standard compound is amplified. The above-mentioned separation may be attained by, for example, washing with a buffer solution.

5-4. Immobilized Antigen Method

In an embodiment where the immobilized antigen method is applied to the immunochromatography kit according to the present invention (hereinafter referred to simply as the immobilized antigen method), the analysis of the analyte may be conducted, for example, in the following procedures though the procedures are not limited thereto. First, a second antibody having specificity for an analyte (antibody) is prepared in advance by the method described above. The second antibody is labeled in advance. The antigen to which the analyte (antibody) binds specifically is immobilized on a suitable insoluble thin film-shaped support (for example, a nitrocellulose membrane, a glass fiber membrane, a nylon membrane or a cellulose membrane) and then brought into contact with a test sample (or its extract) that may possibly contain the analyte (antibody), so that an antigen-antibody reaction occurs if the analyte is present in the sample. This antigen-antibody reaction may be carried out in the same manner as in usual antigen-antibody reaction. During or after the antigen-antibody reaction, the sample is brought into contact further with an excess amount of the labeled second antibody, so that an immune complex of the immobilized antigen-analyte (antibody)-labeled second antibody forms if the analyte is present in the sample.

In the immobilized antigen method, after the completion of the reaction of the immobilized antigen, the analyte (antibody) and the second antibody, the labeled second antibody not involved in the immune complex is removed. Then, for example, metal ions and a reducing agent may be supplied to a region of the insoluble thin film-shaped support on which the immobilized antigen is immobilized, so that the signal from the label of the labeled second antibody involved in the immune complex is amplified. As an alternative, metal ions and a reducing agent may be added to the labeled second antibody, and the labeled second antibody, the metal ions and the reducing agent may be simultaneously added to the thin film-shaped support, so that the signal from the label of the labeled second antibody involved in the immune complex is amplified.

6. Inorganic Silver Salt or Silver Complex

The inorganic silver salt or the silver complex used in the present invention is a compound containing a reducible silver ion. Preferably, the salt or complex is an inorganic silver or a silver complex which forms metallic silver, which is relatively stable against light, when the salt or complex is heated to 50° C. or higher in the presence of a reducing agent.

The inorganic silver salt used in the present invention is, for example, a silver halide (such as silver chloride, silver bromide, silver chlorobromide, silver iodide, silver chloroiodide, silver chloroiodobromide, or silver iodobromide), silver thiosulfate, silver thicyanate, or a silver sulfite.

The inorganic silver salt used in the present invention is preferably a silver halide.

The method for forming particles of the silver halide used in the invention is well known in the photographic industry. For example, methods described in Research Disclosure No. 17029, in June 1978, and U.S. Pat. No. 3,700,458 may be used. Specifically, the silver halide may be prepared by adding, to a solution of a gelatin or some other polymer, a silver supplying compound (for example, silver nitrate) and a halogen supplying compound.

The particle size of the silver halide is preferably very small in order to make examination noise small. Specifically, the size is preferably 0.20 μm or less, more preferably 0.10 μm or less, and even more preferably in the range of nanoparticles. The particle size referred to herein is a diameter of a circular image having the same area as the projected area of the silver halide particle (the projected area of the main plane in the case of a tabular particle).

Silver thiosulfate, silver thiocyanate, and silver sulfite can also be prepared in the same manner as the formation of silver halide particles, by mixing a silver supplying compound (such as silver nitrate) with a silver salt of a thiosulfate (such as a sodium salt, a potassium salt or an ammonium salt), a silver salt of a thiocyanate (such as a sodium salt, a potassium salt or an ammonium salt), and a sulfite (such as a sodium salt, a potassium salt or an ammonium salt), respectively.

Examples of the silver complex used in the present invention include a complex of a thiosulfate and a silver ion, a complex of a thiocyanate and a silver ion, and a composite silver complex thereof, and a complex of a sugar thione derivative and a silver ion, a complex of a cyclic imide compound (such as uracil, urazole, 5-methyluracil, or barbituric acid) and a silver ion, and a complex of a 1,1-bissulfonylalkane and a silver ion. The silver complex used in the invention is preferably a complex of a cyclic imide compound (such as uracil, urazole, 5-methyluracil, or barbituric acid) and a silver ion.

The silver complex used in the invention may be prepared by a generally-known salt forming reaction. For example, the complex may be prepared by mixing in water or a water-miscible solvent a water-soluble silver supplier (such as silver nitrate) and a ligand compound corresponding to the silver complex. The prepared silver complex can be used after salts as byproducts are removed by a known desalting method such as dialysis or ultrafiltration.

The inorganic silver salt or the silver complex is contained generally in an amount of 0.001 to 0.2 mol/m², preferably 0.01 to 0.05 mol/m², in terms of the silver amount.

7. Complexing Agent

The immunochromatography kit according to the invention preferably contains a solubilizing agent for the inorganic silver salt or the silver complex. The solubilizing agent used in the invention is preferably a compound selected from those used as ligands for forming a silver complex described in the above paragraphs for “silver complex”. The compound is, for example, a thiosulfate, a thiocyanate, a sugar thione derivative, a cyclic imide compound, or a 1,1-bissulfonylalkane. The solubilizing agent used in the invention is more preferably a cyclic imide compound such as uracil, urazole, 5-methyluracil, or barbituric acid.

The solubilizing agent used in the invention is used in an amount of preferably 0.1 to 10 moles per mole of silver ions.

8. Reducing Agent

As the reducing agent for silver ion, any material capable of reducing silver(I) ion into silver may be used.

Developing agents (for example, methyl gallate, hydroquinone, substituted hydroquinone, 3-pyrazolidones, p-aminophenols, p-phenylenediamines, hindered phenols, amidoximes, azines, catechols, pyrogallols, ascorbic acid (or derivatives thereof) and leuco dyes) used in wet-process silver halide photosensitive materials, or other materials evident for those skilled in the art (see, for example, U.S. Pat. No. 6,020,117 (Bauer et al.)) may be used in the present invention.

The term “ascorbic acid reducing agent” refers to ascorbic acid or a derivative thereof. Ascorbic acid reducing agents are described in many literatures as described below, including, for example, U.S. Pat. No. 5,236,816 (Purol et al.) and literatures cited therein.

The reducing agent in the present invention is preferably an ascorbic acid reducing agent. Useful ascorbic acid reducing agents include ascorbic acid, analogues thereof, isomers thereof and derivatives thereof. Examples of such compounds include, but are not limited to, D- or L-ascorbic acid and sugar derivatives thereof (for example, sorboascorbic acid, gamma-lactoascorbic acid, 6-desoxy-L-ascorbic acid, L-rhamnoascorbic acid, imino-6-desoxy-L-ascorbic acid, glucoascorbic acid, fucoascorbic acid, glucoheptoascorbic acid, maltoascorbic acid, L-arabosascorbic acid), sodium ascorbate, potassium ascorbate, isoascorbic acid (or L-erythroascorbic acid) and salts thereof (for example, alkali metal salts, ammonium salts or salts known in the art), endiol-containing ascorbic acid, enaminol-containing ascorbic acid, and thioenol-containing ascorbic acid, for example compounds described in U.S. Pat. No. 5,498,511, EP-A 0585,792, EP-A 0573700, EP-A 0588408, U.S. Pat. Nos. 5,089,819, 5,278,035, 5,384,232, 5,376,510, JP 7-56286, U.S. Pat. No. 2,688,549 and Research Disclosure 37152 (March, 1995).

Among these compounds, D-, L-, or D,L-ascorbic acid (and alkali metal salts thereof) and isoascorbic acid (and alkali metal salts thereof) are preferable, and sodium salts are preferable. If necessary, a mixture of two or more such reducing agents may be used.

A hindered phenol may be preferably used singly or in combination with one or more gradation-hardening reducing agents and/or contrast enhancers.

A hindered phenol is a compound having only one hydroxyl group on a benzene ring and at least one substituent at the ortho-position relative to the hydroxyl group. The hindered phenol reducing agent may have plural hydroxyl groups insofar as the hydroxyl groups are located on different benzene rings.

Examples of the hindered phenol reducing agent include binaphthols (that is, dihydroxybinaphthols), biphenols (that is, dihydroxybiphenols), bis(hydroxynaphthyl) methanes, bis(hydroxyphenyl) methanes (i.e., bisphenols), hindered phenols and hindered naphthols, each of which may be substituted.

Typical binaphthols include, but are not limited to, 1,1′-bi-2-naphthol, 1,1′-bi-4-methyl-2-naphthol, 6,6′-dibromo-bi-2-naphthol, and compounds described in U.S. Pat. Nos. 3,094,417 and 5,262,295.

Typical biphenols include, but are not limited to, 2,2′-dihydroxy-3,3′-di-t-butyl-5,5′-dimethylbiphenyl, 2,2′-dihydroxy-3,3′,5,5′-tetra-t-butylbiphenyl, 2,2′-dihydroxy-3,3′-di-t-butyl-5,5′-dichlorobiphenyl, 2-(2-hydroxy-3-t-butyl-5-methylphenyl)-4-methyl-6-n-hexylphenol, 4,4′-dihydroxy-3,3′,5,5′-tetra-t-butylbiphenyl, 4,4′-dihydroxy-3,3′,5,5′-tetramethylbiphenyl, and compounds described in U.S. Pat. No. 5,262,295.

Typical bis(hydroxynaphthyl)methanes include, but are not limited to, 4,4′-methylenebis(2-methyl-1-naphthol) and compounds described in U.S. Pat. No. 5,262,295.

Typical bis(hydroxyphenyl) methanes include, but are not limited to, bis(2-hydroxy-3-t-butyl-5-methylphenyl) methane (CAO-5), 1,1′-bis(2-hydroxy-3,5-dimethylphenyl)-3,5,5-trimethyl hexane (NONOX or PERMANAX WSO), 1,1′-bis(3,5-di-t-butyl-4-hydroxyphenyl) methane, 2,2′-bis(4-hydroxy-3-methylphenyl) propane, 4,4′-ethylidene-bis(2-t-butyl-6-methylphenol), 2,2′-isobutylidene-bis(4,6-dimethylphenol) (LOWINOX 221B46), 2,2′-bis(3,5-dimethyl-4-hydroxyphenyl) propane, and compounds described in U.S. Pat. No. 5,262,295.

Typical hindered phenols include, but are not limited to, 2,6-di-t-butylphenol, 2,6-di-t-butyl-4-methylphenol, 2,4-di-t-butylphenol, 2,6-dichlorophenol, 2,6-dimethylphenol, and 2-t-butyl-6-methylphenol.

Typical hindered naphthols include, but are not limited to, 1-naphthol, 4-methyl-1-naphthol, 4-methoxy-1-naphthol, 4-chloro-1-naphthol, 2-methyl-1-naphthol, and compounds described in U.S. Pat. No. 5,262,295.

Other compounds disclosed as reducing agents include amidoximes (for example, phenylamidoxime), 2-thienylamidoxime, p-phenoxyphenylamidoxime, azines (for example, 4′-hydroxy-3,5-dimethoxybenzaldehydrazine), a combination of an aliphatic carboxylic allyl hydrazide and ascorbic acid (for example, a combination of 2,2′-bis(hydroxymethyl)-propionyl-β-phenyl hydrazide and ascorbic acid), a combination of a polyhydroxybenzene and at least one of hydroxylamine, reductone or hydrazine (for example, a combination of hydroquinone and bis(ethoxyethyl)hydroxylamine), piperidi-4-methylphenylhydrazine, hydroxamic acids (for example, phenylhydroxamic acid, p-hydroxyphenylhydroxamic acid, and o-alaninehydroxamic acid), a combination of an azine and a sulfonamidophenol (for example, a combination of phenothiazine and 2,6-dichloro-4-benzenesulfonamidophenol), α-cyanophenylacetic acid derivatives (for example, ethyl-α-cyano-2-methylphenylacetic acid, ethyl-α-cyanophenylacetic acid), bis-o-naphthol (for example, 2,2′-dihydroxy-1-binaphthyl, 6,6′-dibromo-2,2′-dihydroxy-1,1′-binaphthyl, bis(2-hydroxy-1-naphthyl)methane), a combination of bis-o-naphthol and a 1,3-dihydroxybenzene derivative (for example, 2,4-dihydroxybenzophenone, 2,4-dihydroxyacetophenone), 5-pyrazolones (for example, 3-methyl-1-phenyl-5-pyrazolone), reductones (for example, dimethylaminohexose reductone, anhydrodihydro-aminohexose reductone, or anhydrodihydro-piperidone-hexose reductone), sulfonamidophenol reducing agents (for example, 2,6-dichloro-4-benzenesulfonamidophenol, p-benzenesulfonamidophenol), indane-1,3-diones (for example, 2-phenylindane-1,3-dione), chromans (for example, 2,2-dimethyl-7-t-butyl-6-hydroxychroman), 1,4-dihydroxypyridines (for example, 2,6-dimethoxy-3,5-dicarbetoxy-1,4-dihydropyridine), ascorbic acid derivatives (1-ascorbic palmitate, ascorbic stearate), unsaturated aldehydes (ketones), and 3-pyrazolidones.

As the reducing agent usable in the present invention, substituted hydrazines may be mentioned such as sulfonyl hydrazines described in U.S. Pat. No. 5,464,738. Other useful reducing agents are described in, for example, U.S. Pat. Nos. 3,074,809, 3,094,417, 3,080,254 and 3,887,417. Auxiliary reducing agents descried in U.S. Pat. No. 5,981,151 are also useful.

The reducing agent may be a combination of a hindered phenol reducing agent and one or more compounds selected from various auxiliary reducing agents such as those mentioned below. A mixture of this combination plus a contrast enhancer (that is, a mixture of the 3 components) is also useful. As the auxiliary reducing agent, it is possible to use trityl hydrazide and formyl-phenyl hydrazide described in U.S. Pat. No. 5,496,695.

A contrast enhancer may be used in combination with the reducing agent. Useful contrast enhancers include, but are not limited to, hydroxylamines (including hydroxylamine, alkyl-substituted derivatives thereof and aryl-substituted derivatives thereof), alkanolamines and phthalic ammonium described in U.S. Pat. No. 5,545,505, hydroxamic acid compounds described in U.S. Pat. No. 5,545,507, N-acylhydrazine compounds described in U.S. Pat. No. 5,558,983, and hydrogen atom donor compounds described in U.S. Pat. No. 5,637,449.

Not all combinations of reducing agents and inorganic silver salts or silver complexes are equally effective.

The reducing agent in the present invention may be contained in an amount of 1 mass % to 10 mass % (dry mass) based on the amount of silver. When the reducing agent is added to a layer other than the layer containing the inorganic silver salt or the silver complex in a multilayer structure, the amount of the reducing agent is slightly higher and is desirably from about 2 mass % to about 15 mass %. An auxiliary reducing agent is contained in an amount of about 0.001 mass % to 1.5 mass % (dry weight).

The kit according to the present invention is preferably heated after development of the sample to be examined. The heating temperature is preferably in the range of 40° C. to 90° C., and the heating time is preferably in the range of 1 second to 120 seconds.

Exemplary embodiments of the invention are described below.

<1> An immunochromatography kit including an inorganic silver salt or a silver complex, a reducing agent for silver ions, and a metal colloid label or a metal chalcogenide label, wherein the immunochromatography kit uses an immune reaction of an analyte and an antibody or antigen that can specifically bind to the analyte, and analyzes a signal from the label derived from an immobilized immune complex.

<2> The immunochromatography kit according to item <1>, wherein the inorganic silver salt includes silver halide.

<3> The immunochromatography kit according to item <2>, wherein the silver halide is silver chloride, silver bromide, silver chlorobromide, silver iodide, silver chloroiodide, silver chloroiodobromide or silver iodobromide.

<4> The immunochromatography kit according to item <1>, wherein the silver complex includes a complex that includes a silver ion and a complexing agent selected from the group consisting of a thiosulfate, a thiocyanate, a sulfite, a sugar thione derivative, a cyclic imide and a 1,1-bissulfonylalkane.

<5> The immunochromatography kit according to item <4>, wherein the complexing agent is a cyclic imide compound.

<6> The immunochromatography kit according to item <1>, further including a solvent for the inorganic silver salt or the silver complex.

<7> The immunochromatography kit according to item <6>, wherein the solvent for the inorganic silver salt or the silver complex includes a thiosulfate, a thiocyanate, a sulfite, a sugar thione derivative, a cyclic imide compound or a 1,1-bissulfonylalkane.

<8> The immunochromatography kit according to item <1>, wherein the inorganic silver salt or the silver complex, the reducing agent for silver ions, and the metal colloid label or the metal chalcogenide label are contained in a single component part of the kit.

<9> The immunochromatography kit according to item <8>, wherein the immunochromatography kit includes, in the single component part, a layer containing the inorganic silver salt or the silver complex and the reducing agent for silver ions, and a layer containing the metal colloid label or the metal chalcogenide label.

<10> The immunochromatography kit according to item <1>, wherein the metal colloid is a gold colloid, a silver colloid, a platinum colloid, or a composite colloid thereof.

<11> The immunochromatography kit according to item <10>, wherein the metal colloid has an average particle diameter of 5 to 100 nm.

<12> The immunochromatography kit according to item <1>, wherein the metal chalcogenide is a sulfide, selenide or telluride of gold, silver, platinum, palladium, lead, zinc, cadmium, tin, chromium, copper or cobalt.

<13> The immunochromatography kit according to item <12>, wherein the metal chalcogenide has an average particle diameter of 5 to 100 nm.

<14> The immunochromatography kit according to item <1>, wherein the reducing agent is an ascorbic acid reducing agent.

<15> The immunochromatography kit according to item <1>, further including a sensitized sheet.

<16> The immunochromatography kit according to item <1>, wherein the antibody or antigen is immobilized on a support.

<17> The immunochromatography kit according to item <16>, wherein the immune complex comprises the antibody or antigen, the analyte and an additional labeled antibody or antigen.

<18> The immunochromatography kit according to <16>, wherein the immune complex comprises the antibody or antigen and the analyte, and the analyte is labeled.

### EXAMPLES

### Example 1

In Example 1, it was verified as described below that the immunochromatography kit according to the present invention showed a high sensitivity in an hCG detecting system.

1. Production of a Metal Sulfide Colloid (Detection Label) Modified with an Anti-hCG Antibody

### (Preparation of Palladium Sulfide Colloid)

1000 mL of water, 30 g of glycerin, and palladium chloride and sodium sulfide (molar ratio=1:1) were mixed to form a palladium sulfide colloidal solution having particle diameters of about 30 nm. 1 mL of a 50 mM KH₂PO₄ buffer (pH=7.0) was added to 9 mL of the palladium sulfide colloidal solution so as to adjust the pH. Thereafter, 1 mL of a 50 μg/mL anti-hCG antibody (Anti-hCG 5008 SP-5, manufactured by Medix Biochemical Co.) solution was added thereto, and the resultant solution was stirred. The solution was allowed to stand still for 10 minutes, and then 550 μL of a 1% by mass aqueous solution of polyethylene glycol (PEG, manufactured by Wako Pure Chemical Industries, Ltd., Mw: 20000, product number: 168-11285) was added thereto, followed by stirring. Subsequently, thereto was added 1.1 mL of a 10% by mass aqueous solution of bovine serum albumin (BSA Fraction V, manufactured by Sigma, product number: A-7906), followed by stirring. This solution was centrifuged (himac CF16RX, manufactured by Hitach Ltd.) at 8000 G and 4° C. for 30 minutes, and then the supernatant was removed so that about 1 mL remained. The remaining solution was re-dispersed with an ultrasonic washer. Thereafter, the obtained dispersion was dispersed in 20 mL of a stock solution for a palladium sulfide colloidal solution (20 mM Tris-HCl buffer (pH=8.2), containing 0.05% by mass PEG (Mw: 20000), 150 mM NaCl, 1% by mass BSA, and 0.1% by mass NaN₃). The resultant dispersion was again centrifuged at 8000 G and 4° C. for 30 minutes, and then the supernatant was removed such that about 1 mL remained. The palladium sulfide colloidal solution was re-dispersed with an ultrasonic washer to yield an antibody-modified palladium sulfide colloidal solution (average diameter: 30 nm).

2. Preparation of a Metal Sulfide Antibody Holding Pad

The antibody-modified palladium sulfide colloidal solution prepared in the item 1 was diluted with water and a coating solution for the palladium sulfide colloidal solution (20 mM Tris-Hcl buffer (pH=8.2), containing 0.05% by mass PEG (Mw: 20000) and 5% by mass sucrose) to set the OD at 520 nm to 1.5. This solution was uniformly applied to glass fiber pads (GLASS FIBER CONJUGATE PAD, manufactured by Millipore Corp.) cut in the size of 8 mm×150 mm in an amount of 0.8 mL per pad. The pads were dried under reduced pressure overnight to give palladium sulfide colloidal solution antibody holding pads.

3. Preparation of an Antibody-Immobilized Membrane (Chromatographic Carrier)

An antibody-immobilized membrane was prepared in the following manner by immobilizing an antibody on a nitrocellulose membrane (HIFLOW PLUS HF120 with a plastic lining, manufactured by Millipore) cut in the size of 25 mm×200 mm. Using a coater of inkjet type (BioDot Ltd.), the membrane with one of its long sides directed downwards was coated, at a position of 8 mm from the bottom, with an anti-hCG monoclonal antibody for immobilization (ANTI-ALPHA SUBUNIT 6601 SPR-5, manufactured by Medix Biochemica) prepared at a concentration of 0.5 mg/ml, thereby forming a coating in a line shape with a width of about 1 mm (“detection part”). In a similar manner, the membrane was coated, at a position of 12 mm from the bottom, with a control anti-mouse IgG antibody (anti-mouse IgG (H+L), rabbit F(ab′)2, Product No. 566-70621, Wako Pure Chemical Industries, Ltd.) prepared at a concentration of 0.5 mg/ml, thereby forming a line-shaped coating (“control part”). The coated membrane was dried at 50° C. for 30 minutes with a hot-air dryer. The membrane was immersed in 500 ml of a blocking solution (50 mM borate buffer, pH 8.5, containing 0.5 mass % casein (milk-derived product, Product No. 030-01505, manufactured by Wako Pure Chemical Industries, Ltd.)) in a vat, and was left therein for 30 minutes. Thereafter, the membrane was transferred to and immersed in 500 ml of a washing-stabilizing solution (0.5 mass % sucrose, 0.05 mass % sodium cholate, 50 mM Tris-HCl, pH 7.5) in a similar vat, and was left therein for 30 minutes. The membrane was taken out of the solution, and was dried overnight at room temperature to give an antibody-immobilized membrane.

4. Preparation of a Sensitized Sheet

### 4-1. Preparation of a Coating Material

1) Preparation of a Dispersion of an Uracil Silver Complex in Gelatin

A sodium salt of uracil and silver nitride were mixed at a mol ratio of 2:1 in an aqueous gelatin solution to give an uracil silver complex.

2) An Aqueous Solution of Ascorbic Acid as a Reducing Agent and an Aqueous Solution of a Sodium Salt of Uracil as a Complexing Agent

### 4-2. Application of a Silver-Salt-Containing Layer

A coating solution was obtained by adding the aqueous ascorbic acid solution (in an amount of 1.2 times the mol number of silver) and the aqueous solution of the sodium salt of uracil (in an amount of 1.5 times the mol number of silver) successively to the uracil silver complex dispersion in the gelatin obtained above. This coating solution was applied to a temporary support, to give a silver coating amount of 0.6 g/m². An aqueous gelation solution was applied thereon in an amount of 0.1 g/m² as a protecting layer, followed by drying.

### 4-3. Preparation of a Sensitized Sheet

The silver-containing layer coated sample was cut into a 200 mm×70 mm size. A polyester pressure-sensitive adhesive tape (No 31B 71 HIGH, manufactured by Nitto Denko Corp.) was adhered to the surface of the protecting layer of the coated sample, and then the silver-salt-containing coated layer was peeled off the temporary support by peeling the pressure-sensitive adhesive tape adhered to the coated layer. In this way, a sensitized sheet was obtained.

5. Preparation of Immunochromatography Kits

### 5-1. Fabrication of a Kit A

### Comparative Example

As illustrated in FIG. 2, the antibody immobilized membrane 3 was attached to a back pressure-sensitive adhesive sheet (ARCARE 9020, manufactured by Nippun TechnoClaster Inc.). At this time, among the two long sides of the membrane, the long side at the anti-hCG antibody line side was arranged downwards. The palladium sulfide colloidal antibody holding pad 2 was attached onto the antibody-immobilized membrane such that the palladium sulfide colloidal antibody holding pad 2 overlapped the lower portion of the antibody-immobilized membrane by about 2 mm. Further, a sample addition pad 5 (glass fiber pad (GLASS FIBER CONJUGATE PAD, manufactured by Millipore Corp.) cut in the size of 18 mm×150 mm was attached onto the palladium sulfide colloidal antibody holding pad such that the sample addition pad 5 overlapped the lower portion of the palladium sulfide colloid antibody holding pad by about 4 mm. An absorbent pad 4 (cellulose membrane cut in the size of 20 mm×150 mm (trade name: CELLULOSE FIBER SAMPLE PAD, manufactured by Millipore)) was attached onto the antibody-immobilized membrane such that the absorbent pad 4 overlapped the upper portion of the antibody-immobilized membrane by about 5 mm. Using a guillotine cutter (trade name: CM4000, manufactured by Nippun TechnoClaster Inc.), the resultant laminated member was cut in parallel to the short side of the laminated member such that the long side of the member was cut at 5 mm intervals, whereby immunochromatographic strips of 5 mm×55 mm were prepared. These strips were placed in a plastic case (Nippun TechnoClaster Inc.) to give an immunochromatography kit A for test. The capture site 3a was composed of a detection part 31 for detecting a sample antibody and a control part 32 for indicating a process noise, and judgment can be made from the difference in coloring (darkening) density between these parts. The region of the antibody-immobilized membrane 3 where the anti-hCG monoclonal antibody for immobilization was applied in a line shape was the detection part 31, and the region wherein the control anti-mouse IgG antibody was applied in a line shape was the control part 32.

### 5-2. Fabrication of a Kit B (According to the Invention)

The antibody-immobilized membranes 3 prepared in item 3 above was attached to a black pressure-sensitive adhesive sheet 1 (ARCARE 9020, manufactured by Nippun Techno Claster Inc.). At this time, among the two long sides of the membrane, the long side at the anti-hCG antibody line side was arranged downwards. The sensitized sheet 6 was attached thereon such that the silver-complex-containing layer surface contacts the surface of the antibody-immobilized membrane. The palladium sulfide colloidal antibody holding pad 2 prepared in item 2 above was attached onto the antibody-immobilized membrane such that the pad 2 overlapped the lower portion of the antibody-immobilized membrane by about 2 mm. The sample addition pad 5 (glass fiber pad (GLASS FIBER CONJUGATE PAD, manufactured by Millipore Corp.) cut in the size of 18 mm×150 mm size was attached onto the palladium sulfide colloid antibody holding pad such that the sample addition pad 5 overlapped the lower portion of the palladium sulfide colloid antibody holding pad by about 4 mm. An absorbent pad 4 (cellulose membrane cut in the size of 20 mm×150 mm (trade name: CELLULOSE FIBER SAMPLE PAD, manufactured by Millipore)) was attached onto the antibody-immobilized membrane such that the absorbent pad 4 overlapped the upper portion of the antibody-immobilized membrane by about 5 mm. Using a guillotine cutter (trade name: CM4000, manufactured by Nippun TechnoClaster Inc.), the resultant laminated member was cut in parallel to the short side of the laminated member such that the long side of the member was cut at 5 mm intervals, whereby immunochromatographic strips of 5 mm×55 mm were prepared. These strips were placed in a plastic case (Nippun TechnoClaster Inc.) to give an immunochromatography kit B for test.

6. Performance Evaluation

### 1) Minimum Detectable Sensitivity Testing Method

hCG (trade name: RECOMBINANT HCG R-506 manufactured by Rohto Pharmaceutical Co., Ltd.) was dissolved in a PBS buffer containing 1 mass % BSA to prepare test hCG solutions at several concentrations.

100 μL of the test hCG solution at each concentration was dropped onto each immunochromatography kit for test. 10, 15, 20, 30 and 60 minutes after the dropping, the site (capture site) of the antibody-immobilized membrane on which the anti-hCG antibody had been applied was visually checked to judge the degree of coloration according to the following criteria (4 levels):

darkly colored “+++”;

colored “++”,

slightly colored “+”; and

uncolored “−”.

The lowest concentration at which the detection was possible was assumed to be the minimum detectable sensitivity for the kit.

### 2) Results

TABLE 1

| | |
|---|---|
| | Degree of Coloration in Detection Zone |
| | (15 minutes after the dropping of the test |
| hCG concentration | hCG solution) |

| (ng/mL) | Kit A | Kit B |
|---|---|---|
| 100.00 | +++ | +++ |
| 30.00 | ++ | +++ |
| 10.00 | + | +++ |
| 3.00 | − | +++ |
| 1.00 | − | +++ |
| 0.30 | − | ++ |
| 0.10 | − | ++ |
| 0.03 | − | + |
| 0.01 | − | − |
| 0.00 | − | − |

As is evident from Table 1, the kit B according to the invention was able to detect hCG with extremely high sensitivity as compared with the comparative kit A.

According to the present invention, the sensitivity of immunochromatography can be heightened while retaining an advantage of immunochromatography in simplicity and promptness; in other words, an immunochromatography kit can be provided which enables rapid and easy measurement with higher sensitivity than the measurement with conventionally known immunochromatographic assay kits.

All publications, patent applications, and technical standards mentioned in this specification are herein incorporated by reference to the same extent as if each individual publication, patent application, or technical standard was specifically and individually indicated to be incorporated by reference.

What is claimed is:

1. An immunochromatography kit comprising an inorganic silver salt or a silver complex, a reducing agent for silver ions, and a metal colloid label or a metal chalcogenide label,
wherein the immunochromatography kit uses an immune reaction of an analyte and an antibody or antigen that can specifically bind to the analyte, and analyzes a signal from a label derived from the immobilized immune complex.

2. The immunochromatography kit according to claim 1, wherein the inorganic silver salt comprises a silver halide.

3. The immunochromatography kit according to claim 2, wherein the silver halide is silver chloride, silver bromide, silver chlorobromide, silver iodide, silver chloroiodide, silver chloroiodobromide or silver iodobromide.

4. The immunochromatography kit according to claim 1, wherein the silver complex comprises a complex of a silver ion and a complexing agent selected from the group consisting of a thiosulfate, a thiocyanate, a sulfite, a sugar thione derivative, a cyclic imide compound and a 1,1-bissulfonylalkane.

5. The immunochromatography kit according to claim 4, wherein the complexing agent is a cyclic imide compound.

6. The immunochromatography kit according to claim 1, further comprising a solubilizing agent for the inorganic silver salt or the silver complex.

7. The immunochromatography kit according to claim 6, wherein the solubilizing agent for the inorganic silver salt or the silver complex comprises a thiosulfate, a thiocyanate, a sulfite, a sugar thione derivative, a cyclic imide compound or a 1,1-bissulfonylalkane.

8. The immunochromatography kit according to claim 1, wherein the immunochromatography kit comprises, in a single component part of the kit, the inorganic silver salt or the silver complex, the reducing agent for silver ions, and the metal colloid label or the metal chalcogenide label.

9. The immunochromatography kit according to claim 8, wherein the immunochromatography kit comprises, in the single component part of the kit, a layer containing the inorganic silver salt or the silver complex and the reducing agent for silver ions, and a layer containing the metal colloid label or the metal chalcogenide label.

10. The immunochromatography kit according to claim 1, wherein the metal colloid is a gold colloid, a silver colloid, a platinum colloid, or a composite colloid thereof.

11. The immunochromatography kit according to claim 10, wherein the metal colloid has an average particle diameter of 5 to 100 nm.

12. The immunochromatography kit according to claim 1, wherein the metal chalcogenide is a sulfide, selenide or telluride of gold, silver, platinum, palladium, lead, zinc, cadmium, tin, chromium, copper or cobalt.

13. The immunochromatography kit according to claim 12, wherein the metal chalcogenide has an average particle diameter of 5 to 100 nm.

14. The immunochromatography kit according to claim 1, wherein the reducing agent is an ascorbic acid reducing agent.

15. The immunochromatography kit according to claim 1, further comprising a sensitized sheet.

16. The immunochromatography kit according to claim 1, wherein the antibody or antigen is immobilized on a support.

17. The immunochromatography kit according to claim 16, wherein the immune complex comprises the antibody or antigen, the analyte and an additional labeled antibody or antigen.

18. The immunochromatography kit according to claim 16, wherein the immune complex comprises the antibody or antigen and the analyte, and the analyte is labeled.
